(19) United States
(12) Patent Application Publication
(10) Pub. No.: US 2011/0008857 A1
(43) Pub. Date: Jan. 13, 2011

(54) Method for Producing Polyhydroxyalkanoate

(75) Inventors: Kevin O'CONNOR, Malahide (IE); Jasmina Nikodinovic, Dublin (IE)

Correspondence Address: GREENLEE SULLIVAN P.C., 4875 PEARL EAST CIRCLE, SUITE 200, BOULDER, CO, 80301, US

(73) Assignee: University College Dublin, National University of Ireland, Dublin, Dublin (IE)

(21) Appl. No.: 12/501,339

(22) Filed: Jul. 10, 2009

(51) Int. Cl. C12P 7/44

(52) U.S. Cl. 435/142

(57) ABSTRACT

A method for producing polyhydroxyalkanoate (PHA), comprising (i) culturing in a culture medium containing a substrate comprising one or more monoaromatic hydrocarbons selected from the group consisting of benzene, toluene, ethylbenzene, ortho-xylene, meta-xylene, para-xylene and styrene, one or more strains of Pseudomonas putida selected from the group consisting of F1, mt-2 and CA-3 having the respective accession numbers DSM 6899, NCIMB10432 and NCIMB41162; and (ii) recovering the PHA produced from the culture medium; with the proviso that the substrate cannot comprise styrene in the absence of at least one of benzene, toluene, ethyl benzene and ortho-, meta- or para-xylene.

### CROSS REFERENCE TO RELATED APPLICATIONS

Not applicable.

### BACKGROUND

The present invention relates to a method for producing polyhydroxyalkanoate (PHA) from one or more monoaromatic hydrocarbons selected from benzene, toluene, ethylbenzene, ortho-, meta- or para-xylene and styrene.

Petrochemical non-oxygenated monoaromatic hydrocarbons, in particular benzene, toluene, ethylbenzene and ortho-, meta- and para-xylenes (BTEX compounds) are widely used in the industry as solvents and as starting materials for the production of pharmaceuticals, polymers and paints. Indeed BTEX compounds are among the top 50 chemicals produced and used worldwide. As a consequence of their wide usage, they are common waste materials from industry. BTEX compounds are also major components of oils (approximately 56%, v/v) generated from the heat treatment (pyrolysis) of mixed plastic waste. This oil also contains styrene in high amounts (approximately 19%, v/v). When styrene is present, the BTEX compounds are referred to as BTEXS compounds. BTEXS compounds can be easily separated from the other components of the pyrolysis oil for recycling by existing distillation systems in petrochemical plants, to be degraded further.

The metabolism of BTEXS compounds by microorganisms is well known with physiological, biochemical and molecular investigations of their degradation having been carried out. However, the focus over the last 3 to 4 decades has been on BTEXS biodegradation, and not on their conversion to valuable end products. Typical end products obtained from degradation of BTEXS compounds whether by chemical or microbiological means include carbon dioxide and water which have limited application.

PHA is the general term for a range of diverse biodegradable polymers that consist of polyesters of (R)-3-hydroxyalkanoic acids. These polymers are of interest due to a broad range of applications and the fact that they are completely biodegradable thus offering little or no long term waste issues. These polymers can be accumulated by some bacteria intracellularly as carbon storage materials. It has been shown that PHA accumulation occurs in response to a range of environmental stress factors such as inorganic nutrient limitation. The substrates that are supplied to bacteria to accumulate PHA are divided into two groups 1) PHA related substrates, i.e. alkanoic acids (fatty acids) that resemble the monomers that make up PHA ((R)-3-hydroxyalkanoic acids) and 2) PHA unrelated substrates, which are substrates that do not resemble the monomers that make up PHA e.g. glucose. The conversion of polystyrene to PHA by pyrolysis and feeding of the resultant styrene oil to P. putida CA-3 has previously been demonstrated by Ward et al, 2006.

### SUMMARY OF THE INVENTION

It is an object of the invention to mitigate or eliminate the disadvantages associated with the degradation of BTEXS compounds.

It is a further object of the invention to provide a method for producing PHA from one or more BTEXS compounds.

According to the invention, there is provided a method for producing polyhydroxyalkanoate (PHA), comprising:
(i) culturing in a culture medium containing a substrate comprising one or more monoaromatic hydrocarbons selected from the group consisting of benzene, toluene, ethylbenzene, ortho-xylene, meta-xylene, para-xylene and styrene, one or more strains of Pseudomonas putida selected from the group consisting of F1, mt-2 and CA-3 having the respective accession numbers DSM 6899, NCIMB10432 and NCIMB41162; and
(ii) recovering the PHA produced from the culture medium;
with the following provisos:
   (A) when the substrate comprises benzene, the Pseudomonas putida strain F1 is present in the culture medium;
   (B) when the substrate comprises toluene, the Pseudomonas putida strain(s) F1 and/or mt-2 is/are present in the culture medium;
   (C) when the substrate comprises ethylbenzene, the Pseudomonas putida strain F1 is present in the culture medium;
   (D) when the substrate comprises ortho-, meta- or para-xylene, the Pseudomonas putida strain mt-2 is present in the culture medium;
   (E) when the substrate comprises styrene, the Pseudomonas putida strain CA-3 is present in the culture medium; and
   (F) the substrate cannot comprise styrene in the absence of at least one of benzene, toluene, ethylbenzene and ortho-, meta- or para-xylene.

Accordingly, when the substrate comprises benzene, the one or more strains of Pseudomonas putida comprises F1. When the substrate comprises toluene, the one or more strains of Pseudomonas putida comprise F1 and/or mt-2, preferably F1 and mt-2. When the substrate comprises ortho-xylene, the one or more strains of Pseudomonas putida comprises mt-2. When the substrate comprises meta-xylene, the one or more strains of Pseudomonas putida comprises mt-2. When the substrate comprises para-xylene, the one or more strains of Pseudomonas putida comprises mt-2. Furthermore, when the substrate comprises styrene, the one or more strains of Pseudomonas putida comprises CA-3.

The substrate may comprise any one of or any combination of benzene, toluene, ethylbenzene, ortho-xylene, meta-xylene, para-xylene and styrene, provided that styrene is not used alone. The substrate preferably comprises benzene, toluene, ethylbenzene, a xylene, and styrene, more preferably benzene, toluene, ethylbenzene, para-xylene, and styrene, wherein the one or more strains of Pseudomonas putida preferably comprise F1, mt-2 and CA-3. More preferably, the substrate comprises benzene, toluene, ethylbenzene, para-xylene, and styrene in a respective ratio by weight of total substrate of about 10-20:5-15:1-2:0.5-1.5:10-20; even more preferably in a respective ratio by weight of total substrate of about 16:11:1.5:1:15.

The PHA recovered from the culture medium advantageously comprises at least 80%, preferably at least 85%, more preferably at least 90%, most preferably at least 95% medium chain length (mcl) PHA. MclPHA is classified as comprising repeating units derived from 3-hydroxyalkanoic acid monomers containing 6 carbon atoms (C6) to 14 carbon atoms (C14). Preferably, at least 80%, more preferably 85%, even more preferably at least 90%, most preferably at least 95% of the mclPHA comprises repeating units of C8, C10 and C12 monomers. More preferably, the mclPHA comprises repeating units of C8, C10 and C12 monomers in a respective amount by weight of mclPHA of 10-25% C8, 60%-75% C10 and 10%-25% C12, wherein the amount of C8, C10 and C12 totals 100%. Using the method of the invention, substantially the same respective amounts of C8, C10 and C12 can be obtained using the method of the invention, irrespective of the substrate(s) used.

In a particularly preferred embodiment, the mclPHA recovered comprises repeating units of C8, C10 and C12 monomers in a respective amount by weight of mclPHA of 16% C8, 66% C10 and 18% C12.

When two or more substrates are used, a synergistic effect is shown in terms of the amount of PHA produced, as demonstrated by the results discussed below.

### DESCRIPTION OF THE DRAWINGS

FIG. 1. PHA accumulation by Pseudomonas putida strains from petrochemical monoaromatic hydrocarbons (BTEXS). Pure cultures were grown in E2 mineral medium under nitrogen-limiting conditions (67 mg N/l) in the presence of a single carbon source for 48 h. All data are the average of at least three independent experiments.

FIG. 2. PHA accumulation (bars) and mixed culture population analysis when supplied with a mixture of monoaromatic hydrocarbons. Total cfu/ml (♦), cfu/ml for P. putida CA-3 (▴), P. putida F1 (▪) and P. putida mt-2 (). Mixed cultures were grown for 48 h in E2 mineral medium under nitrogen-limiting conditions (67 mg N/l). All data are the average of at least three independent experiments.

### DETAILED DESCRIPTION OF THE INVENTION

PHAs are generally classified as short chain length PHAs (sclPHAs), medium chain length PHAs (mclPHAs) or long chain length PHAs (IclPHAs), depending upon the number of carbon atoms of the constituting monomers thereof. SclPHA comprises monomers of C3-C5, mclPHA comprises monomers of C6-C14, and IclPHA comprises monomers of more than 14 carbons (>C14). This variation in monomer chain length gives rise to different properties in the polymer, with sclPHAs and IclPHAs both having undesirable properties, sclPHAs having a high degree of crystallinity and being rigid and brittle, and IclPHAs being sticky and very difficult to handle. The properties of sclPHAs and IclPHAs limit the range of their applications. MclPHAs have much more desirable properties, being elastomers with a low degree of crystallinity and a low glass transition temperature and having properties being suitable to a wide range of applications including biodegradable rubber and coating materials. The production of mclPHA according to the invention advantageously provides a polymer which has a wide range of applications and is biodegradable.

The method of the invention preferably comprises culturing the one or more strains of Pseudomonas putida in the culture medium for a period of from about 12 hours to about 72 hours, more preferably from about 24 hours to about 60 hours, most preferably about 48 hours, at a temperature of from about 25° C. to about 35° C., preferably about 30° C.

The culture medium is preferably nitrogen limited, with a maximum nitrogen content of about 0.5 g/liter, more preferably about 0.067 g/liter culture medium. A suitable nitrogen source is sodium ammonium phosphate (NaNH₄HPO₄.4H₂O).

The PHA may be recovered from the culture medium by centrifugation of the culture medium at 4000 rpm for about 20 minutes, followed by washing, preferably with 1 ml of 50 mM phosphate buffer (pH 7.4), and freeze drying.

### Materials and Methods

### Chemicals and Materials

Benzene, toluene, ethylbenzene, p-xylene, styrene, benzoic acid and (R)-3-hydroxyalkanoic acids were analytical grade and supplied from Sigma-Aldrich (Steinheim, Germany). Chloroform, methanol and other solvents were of GC grade and supplied from Fluka (Buchs, Switzerland). A synthetic mixture composed of benzene, toluene, ethylbenzene, p-xylene and styrene in a ratio of 16:11:1.5:1:15 was used to mimic the aromatic components of a mixed plastic pyrolysis sample.

### Microorganisms

Reference Pseudomonas strains were obtained from the Deutsche Sammlung von Mikroorganismen und Zellculturen GmbH (DSMZ), lnhoffenstraβe 7 B, 38124 Braunschweig, Germany or NCIMB culture collection (Aberdeen, Scotland, UK) for their ability to degrade aromatic hydrocarbons. P. putida F1 (DSM 6899) is a benzene, toluene and ethylbenzene degrader; P. putida mt-2 (NCIMB10432) is a toluene and m- and p-xylene degrader; and P. putida CA-3 (NCIMB41162) is a styrene degrader. P. putida F1 (DSM 6899) and P. putida mt-2 (NCIMB10432) are deposited in open collections and are publicly available. P. putida CA-3 (NCIMB41162) was deposited as a patent deposit at the NCIMB institution in Aberdeen, Scotland, on Feb. 21, 2003; this patent deposit will be maintained and replaced if necessary for thirty years or five years after the most recent request, and all restrictions on access to this patent deposit will be removed with the grant of a United States patent referencing this strain. The strains used were found to be not only capable of degrading the monoaromatic hydrocarbons, but were also conveniently found to be capable of accumulating PHA from the monoaromatic hydrocarbons, as described below.

### Culture Conditions for Growth and PHA Accumulation by Bacterial Cultures

Strains were grown in mineral E2 broth (per liter: 3.5 gNaNH₄HPO₄.4H₂O, 7.5 g K₂HPO₄.3H₂O, 3.7 g KH₂PO₄, 0.25 g MgSO₄.7H₂O, 2.78 g FeSO₄.7H₂O, 1.98 g MnCl₂.4H₂O, 2.81 g CoSO₄.7H₂O, 1.47 g CaCl₂.2H₂O, 0.17 g CuCl₂.2H₂O, 0.29 g ZnSO₄.7H₂O; for limited nitrogen conditions, 1.0 g/l of NaNH₄HPO₄.4H₂O was used) and were stored frozen at −20° C. in 15% glycerol/E2 medium stocks. The utilization by the bacterial isolates of the single BTEXS compounds as a sole carbon and energy source was determined by growth on E2 mineral medium plates solidified with 1% (w/v) agarose (Sigma) and supplemented via the vapor phase by placing 40 μl of benzene, toluene, ethylbenzene, styrene, or p-xylene into Eppendorf pipette tips inside the Petri dishes. After 2-4 days of incubation at 30° C. plates were screened for presence of colonies. Growth was confirmed by comparison with control plates without substrate and 20 mM glucose as carbon source respectively.

Single colonies of Pseudomonas putida strains grown on solid media were transferred to 3 ml of E2 media (pH 7.0) containing 20 mM benzoic acid as a carbon source and grown overnight at 30° C. Conical flasks (250 ml) containing 50 ml of E2 liquid media containing 1 g/l sodium ammonium phosphate (NaNH₄HPO₄.4 H₂O) (nitrogen-limited) were then inoculated with bacterial cells (1% inoculum). Corresponding BTEXS compound (350 μl) or a mixture (350 μl) was placed into a central column and flasks were tightly closed using sterile cotton plugs and triple layer of aluminum foil. Cultures were grown shaking at 200 rpm in an incubator at 30° C. for 48 h during which time 350 μl partitioned from the central column into the air and subsequently into the liquid medium where it is utilized by the bacteria.

Analysis of Mixed Culture Growth (Colony Forming units/ml) When Supplied with Mixed Substrates

The colony forming units of various Pseudomonas strains when supplied with the synthetic mixed substrate (BTEXS) were determined over 48 hours by periodically sampling from the liquid culture in shake flasks. The total viable cell count was determined by plating culture dilutions onto LB solid medium, as follows. 100 μl sample was added to 900 μl of sterile phosphate buffer (50 mM, pH 7.4) and mixed by inversion, and this was repeated 5-6 times in order to obtain dilution of the original culture of 1×10⁻⁶ or 1×10⁻⁷. 50 μl of both of these dilutions was plated onto agar plates supplemented with the appropriate carbon source. The specific cell count was determined by plating culture dilutions onto E2 plates supplemented with ethylbenzene for P. putida F1, p-xylene for P. putida mt-2, and styrene for P. putida CA-3. Plates were incubated for 4 days at 30° C. before the enumeration of the colonies.

PHA Extraction from Bacterial Cultures

After 48 h incubation at 30° C. cells were then harvested by centrifugation at 4000 rpm for 20 min in benchtop 5810R centrifuge (Eppendorf). The pellet was washed twice with 1 ml of 50 mM phosphate buffer (pH 7.4) and freeze dried. To determine the polymer content of lyophilized whole cells, approximately 5 mg of the cells was subjected to acidic methanolysis according to the following protocol. Cell material (5-10 mg) or PHA standard was resuspended in 2 ml acidified methanol (15% H₂SO₄, v/v) and 2 ml of chloroform containing 6 mg/l benzoate methyl ester as an internal standard. The mixture was placed in 15 ml Pyrex test tubes and incubated at 100° C. for 3 h (with frequent inversions). The solution was extracted with 1 ml of water (vigorous vortex 2 min). The phases were allowed to separate before removing the top layer (water). The organic phase (bottom layer) was dried with Na₂SO₄ before further analysis.

### Gas Chromatography Analysis

The 3-hydroxyalkanoic acid methyl esters were assayed by gas chromatography (GC) using Hewlett-Packard HP6890 chromatograph equipped with a HP-1 capillary column (30 m by 0.25 mm, 0.25 μm film thickness; J & W Scientific) and a flame ionization detector (FID). A temperature program was used to separate the different 3-hydroxyalkanoic acid methyl esters (60° C. for 3 min; temperature ramp of 5° C. per min; 200° C. for 1 min). For the peak identification, methyl esters of 3-hydroxyalkanoic acid were prepared in similar manner and PHA standards from P. putida CA-3 were used.

The following examples serve to illustrate the invention but it will be appreciated that the invention is not limited to these examples.

### Examples

### Example 1A

### PHA Accumulation from Single BTEXS Compounds by Single Cultures

P. putida strains were examined for their ability to grow and accumulate PHA from a variety of monoaromatic hydrocarbons, and the results are shown in Table 1 below.

TABLE 1
PHA accumulation from benzene, toluene, ethylbenzene, p-xylene, and styrene (BTEXS),
by Pseudomonas putida strains.

| | P. putida strain | | | | | |
|---|---|---|---|---|---|---|
| | F1 | | | mt-2 | | |
| Substrate | CDW (g/l) | PHA (g/l) | C₈:C₁₀:C₁₂^{a} | CDW (g/l) | PHA (g/l) | C₈:C₁₀:C₁₂ |
| Benzene | 0.34 ± 0.03 | 0.034 ± 0.001 | 18:67:15 | NG | | |
| Toluene | 0.72 ± 0.11 | 0.14 ± 0.02 | 20:63:17 | 0.37 ± 0.03 | 0.063 ± 0.003 | 19:65:16 |
| Ethylbenzene | 0.67 ± 0.04 | 0.074 ± 0.001 | 19:65:16 | NG | | |
| p-xylene | NG | | | 0.53 ± 0.01 | 0.12 ± 0.02 | 18:67:15 |
| Styrene | NG | | | NG | | |
| BTEXS mix | | | | | | |
| | P. putida strain | | | | | |
| | CA-3 | | | F1 + mt-2 + CA-3 | | |
| Substrate | CDW (g/l) | PHA (g/l) | C₈:C₁₀:C₁₂ | CDW (g/l) | PHA (g/l) | C₈:C₁₀:C₁₂ |
| Benzene | NG | | | | | |
| Toluene | NG | | | | | |
| Ethylbenzene | NG | | | | | |
| p-xylene | NG | | | | | |
| Styrene | 0.79 ± 0.02 | 0.22 ± 0.04 | 19:66:15 | | | |
| BTEXS mix | | | | 1.03 ± 0.04 | 0.21 ± 0.06 | 16:66:18 |

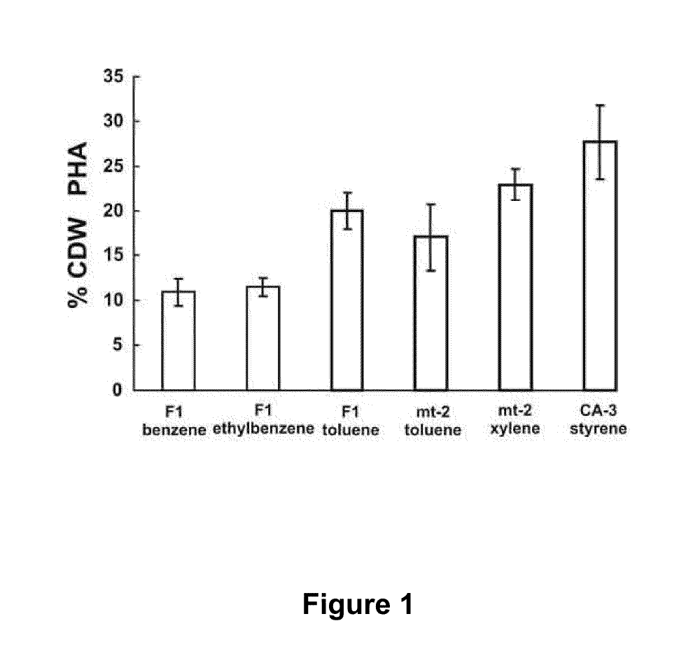

^{a}Traces of C₆ monomer were also detected, but these were not accounted for in the calculation of the total yield of the polymer.
NG = no growth.
Cell yield is given as cell dry weight (CDW); the PHA yield is calculated by multiplying the cell yield (g/l) by the PHA content (% cell dry weight) of the cells; (the results shown in FIG. 1 are obtained by dividing PHA g/l by CDW g/l). Monomeric unit composition of PHA is given as percentage ratio by weight of mcIPHA of C₈:C₁₀:C₁₂. All data is an average of 3 independent measurements.

P. putida F1 when supplied with 350 μl of toluene, benzene or ethylbenzene accumulated PHA to 20%, 12% and 11% of CDW respectively (FIG. 1), under nitrogen limitation (67 mg N/l). However the strains failed to grow with p-xylene or styrene as a sole source of carbon and energy. P. putida F1 achieved a similar cell dry weight when supplied with either toluene or ethylbenzene as the sole source of carbon and energy (Table 1). A 2 fold lower cell dry weight was achieved with benzene as the carbon source. Thus the PHA productivity (g/l) was 4 fold and 2.2 fold higher for toluene and ethylbenzene grown cells respectively compared to benzene grown cells (Table 1). The PHA accumulated by strain F1 was almost identical from all three aromatic carbon sources supplied. The PHA was composed predominantly of 3-hydroxydecanoic acid with 3-hydroxyoctanoic acid and 3-hydroxydodecanoic acid occurring at similar levels (Table 1).

P. putida mt-2 when supplied with 350 μl toluene or p-xylene accumulated PHA up to 18% and 23% of CDW respectively. Strain mt-2 failed to grow with either benzene, ethylbenzene, or styrene as sole carbon and energy source. The cell dry weight achieved by P. putida mt-2 when supplied with toluene was almost half that achieved by P. putida F1 and thus despite a similar PHA content per cell the PHA productivity was 2.2 fold lower for P. putida mt-2. P. putida mt-2 achieves a higher cell dry weight and PHA content per cell dry weight with p-xylene as the substrate compared to toluene (Table 1, FIG. 1). Thus a 1.9 fold higher PHA productivity was achieved with this substrate compared to toluene.

P. putida CA-3 is a known styrene degrader capable of accumulating PHA from styrene (Ward et al 2006). This strain however could not metabolize any other aromatic hydrocarbon investigated in this study (Table 1).

### Example 1B

### PHA Accumulation from BTEXS Mixture by Mixed Culture

A mixed culture was tested to investigate whether it would grow and accumulate PHA when supplied with a mixture of the aromatic compounds, even though it was known that the presence of compounds that are not metabolized by a bacterium may cause toxic effects and hinder growth of that bacterium (Reardon et al, 2000). Additionally, Arvin et al, 1989, reported an antagonistic effect on benzene degradation in the presence of toluene and p-xylene. The negative effects of a non metabolizable substrate (e.g. p-xylene) on bacterial biomass (strain B1) have been reported by Chang and co-workers where toluene is the growth substrate. Batch tests using paired substrates (BT, TX, or BX) revealed competitive inhibition and co-metabolic degradation patterns. Non metabolizable substrates may be partially transformed and the intermediates can inhibit the growth of strains (Munoz, 2007).

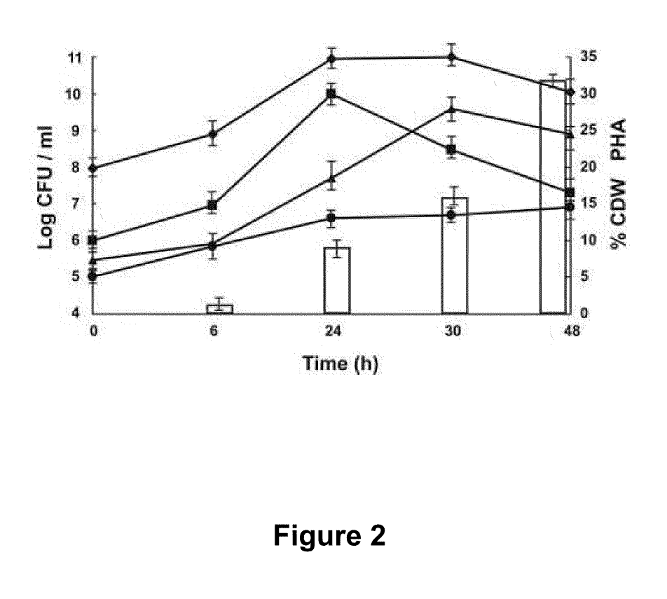

The ability of a defined mixed culture of P. putida F1, mt-2 and CA-3 (1×10⁵ to 1×10⁶ CFU/ml of each and a total 1% inoculum) to metabolize a mixture (350 μl) of aromatic hydrocarbons (benzene, toluene, ethylbenzene, p-xylene, and styrene in a ratio of 16:11:1.5:1:15) was examined with a view to assessing PHA accumulation. The ratio of each substrate to the other was based on the composition of a typical mixed plastic pyrolysis oil. The final cell dry weight achieved after 48 hours of growth was 1.03 g/l. 20.4% of the cell dry weight was composed of PHA representing a PHA productivity of 0.21 g/l (Table 1). The ratio of PHA monomers was almost identical to that achieved by individual strains with individual substrates (Table 1). An analysis of PHA accumulation over the 48 hour growth period showed PHA was detected at low levels after 6 hours of growth and increased ten fold after 24 hours. A further 1.5 and 3 fold increase in PHA production was observed over the next 6 and 24 hours respectively (FIG. 2).

To determine the fate of each of the individual strains in this mixed culture samples were taken periodically over the 48 hour growth period and analyzed for total colony forming units (TCFU) on LB as well as CFU for each individual strain on selected aromatic carbon substrates (see materials and methods) (FIG. 2). The total CFU/ml increased from 9×10⁶ to 9×10¹⁰ over the first 24 hours of growth but did not increase after this time point. Indeed TCFU/ml dropped between 30 hours and 48 hours despite an increase in PHA accumulation over this period (FIG. 2). P. putida F1 is the predominant strain in this mixed culture for the first 24 hours of growth. However the CFU/ml for this organism decreased approximately 1000 fold over the next 24 hours. P. putida mt-2 and CA-3 had identical CFU/ml 6 hours after inoculation. However, P. putida CA-3 outgrew strain mt-2 and was the predominant organism in the mixed culture after 30 hours of growth. P. putida F1 and mt-2 had similar CFU/ml at 48 hours (FIG. 2). Interestingly, at 48 hours, the CFU/ml of P. putida F1 and mt-2 grown on individual substrates was 10 to 40 fold lower than that achieved with mixed substrates showing a synergistic effect when more than two substrates were used (Table 2). P. putida CA-3, which can only utilize styrene as a carbon source, achieved the same CFU/ml when grown in the mixed culture with mixed substrates as when grown as a single culture with styrene as a single substrate.

The investigation of aromatic hydrocarbon biodegradation has often focused on the single substrates alone, neglecting the fact that these compounds rarely occur alone as either a waste or as a pollutant. A synthetic mixture of aromatic hydrocarbons composed of benzene, toluene, ethylbenzene, p-xylene and styrene in the proportions found in the pyrolysis oil of the mixed plastic waste was used. Recently, thermal decomposition of municipal plastic waste (pyrolysis) has received much attention as part of the efforts for environmentally sound sustainable plastic recycling. The obtained oil can be burnt for energy but could also be used as a feedstock for bacteria to make PHA. This principle has been demonstrated with polystyrene where pyrolysis combined with bacterial fermentation has resulted in the production of PHA from polystyrene (Ward et al. 2006). P. putida CA-3 was included in the mixed culture as the other two cultures (F1 and mt-2) are not capable of growth with styrene. 350 μl of a mixture of benzene, toluene, ethylbenzene, p-xylene, and styrene (ratio 16:11:1.5:1:15) was placed in the central glass column in the shake flask containing 50 ml of liquid medium. The volatile liquid partitions into the air and subsequently into the liquid where the bacteria utilize the mixture of compounds as carbon an energy sources. 334.5 mg substrate is present in the 350 μl mixture. A total of 51.5 mg of cell dry weight (1.03 mg/ml) was achieved by the mixed culture supplied with BTEXS. This equates to a growth yield of approximately 17% (g CDW/g substrate supplied). It is hypothesized that this figure may have been even higher but for the fact that the volatile substrates absorb onto the cotton wool seal in the flask and thus not all the substrate is available to the bacteria. This was also observed in previous studies with P. putida CA-3 supplied with styrene in the same way. The supply of volatile substrates in a bioreactor generates much higher cell growth yields due to much more efficient feeding of the substrate to the liquid medium (Ward et al., 2006) and thus it is predicted that higher cell growth yields would be achieved when these strains are grown in a bioreactor.

A higher overall biomass was achieved by mixed cultures (51.5 mg (1.03 mg/ml)) from BTEXS (307 mg substrate) compared to individual strains (e.g. 36 mg (F1), 26.5 mg (mt-2)) supplied with individual substrates (e.g. toluene 303 mg, p-xylene 301 mg) (Table 1), demonstrating a synergistic effect which is discussed below in Example 2. Thus an overall higher PHA productivity was achieved by mixed cultures compared to P. putida F1 and mt-2 alone. The monomer composition of PHA accumulated by all strains was advantageously very similar (Table 1). The monomer composition of the PHA did not change when the strains were switched from growth on a single substrate to the aromatic mixture. This is advantageous for the further process development and for the future application of this technology to different substrate mixtures.

The conversion of petrochemical aromatic hydrocarbons (BTEXS) to mcl-PHA by Pseudomonas species either as single strains or defined mixed cultures has been demonstrated. The ability to convert BTEXS compounds to valuable PHA opens up possibilities for converting mixed plastic waste as an inexpensive substrate to value added products.

### Example 2A

### PHA Accumulation from a Single BTEXS Compound (Toluene) by Single and Mixed Cultures

### (i) CDW Values

In each of the experiments in Table 1, the amount of toluene supplied was 350 μl.

From the 350 μl supplied to strain mt-2, 0.37 g of biomass per liter (g/l) was achieved. Strain F1 grows much better with a biomass of 0.72 g/l. When mt-2 and F1 are grown together they both do not have exclusive access to the 350 μl of toluene but rather they have to share that 350 μl of toluene.

It is hypothesized that several scenarios may arise: mt-2 and F1 both use half the toluene each which means that strain mt-2 uses 175 μl and F1 uses the other 175 μl. This gives rise to mt-2 with a biomass of 0.185 g/l (half of 0.37: see Table 1) and strain F1 with a biomass of 0.36 g/l. This total is 0.545 g/l. Alternatively F1 may out compete mt-2 completely and grow to 0.72 with mt-2 not growing at all. However sampling from the mixed culture (P. putida CA-3, mt-2 and F1) grown on BTEXS showed that mt-2 and F1 were growing in the flask with the BTEXS mixture (FIG. 1) with F1 cell numbers twice that of mt-2 after 24 hours of growth (FIG. 2). Thus it is reasonable to assume that when supplied with toluene alone mt-2 and F1 both grow. By this rationale the maximum biomass achievable by mt-2 and F1 (based solely on toluene supply of 350 μl) is between 0.545 and 0.72. Based on FIG. 2 a ratio of 2 F1 cells to every 1 mt-2 cell would yield a biomass of 0.603 (0.48 g/l F1 and 0.123 g/l mt-2). The actual biomass achieved using a mixture of strains, mt-2 and F1, is shown in Table 2, i.e. 0.78 showing synergy.

TABLE 2

| Strain | Substrate (μl) volume | CDW (g/l) observed |
|---|---|---|
| P. putida mt-2 | 350 | 0.37 ± 0.03 |
| P. putida F1 | 350 | 0.72 ± 0.11 |
| P. putida mt-2 and F1 | 350 | 0.78 ± 0.09 |

### (ii) PHA Production

Again for PHA production when grown alone mt-2 and F1 have access to the full 350 μl of toluene. They must share the toluene when grown as a mixed culture. Given an equal sharing of the toluene then the maximum PHA achievable by mt-2 and F1 is 0.0315 g/l and 0.07 g/l respectively (Total 0.102 g/l). Based on a 2 to 1 ratio for cell number then mt-2 will produce 0.021 g PHA/I and F1 0.093 g PHA/I (Total 00.114 g/l) As shown in Table 3, below, the actual value achieved was 0.19 g/l indicating synergy.

TABLE 3

| Strain | Substrate volume (μl) | PHA (g/l) observed |
|---|---|---|
| P. putida mt-2 | 350 | 0.063 ± 0.003 |
| P. putida F1 | 350 | 0.14 ± 0.02 |
| P. putida mt-2 and F1 | 350 | 0.19 ± 0.04 |

These results shown in Tables 2 and 3 confirm the synergistic effect on biomass and PHA yield when growing a defined mixed culture of Pseudomonas strains (P. putida mt-2 and F1) on a single substrate (toluene), compared with using a single strain.

### Example 2B

### PHA Accumulation from BTEXS Mixture by Mixed Culture

In each of the experiments 350 μl of BTEXS mixture was supplied, comprising 126 μl benzene, 87 toluene μl, 11 μl ethylbenzene, 8 μl p-xylene, and 118 μl styrene.

### (i) CDW Values

In the BTEXS mixture specific substrates can be utilized by certain strains. For example, and referring to the results shown in Table 4, when P. putida mt-2 is grown with the BTEXS mixture it has available to it 87 μl of toluene and 8 μl of p-xylene. However, when grown together with P. putida F1 it has to compete for the toluene and thus its maximum achievable biomass will be less than 0.28 g/l. Similarly the maximum achievable biomass for F1 will also be decreased. P. putida CA-3 will not have to compete with other strains for styrene utilization as it is the only strain capable of growing with styrene as a substrate.

TABLE 4

| | Substrate (μl) | CDW (g/l) |
|---|---|---|
| P. putida mt-2 | 350 | 0.28 ± 0.05 |
| P. putida F1 | 350 | 0.21 ± 0.08 |
| P. putida CA-3 | 350 | 0.48 ± 0.09 |
| P. putida mt-2, F1 and CA-3 | 350 | 1.03 ± 0.04 |

The information shown in Table 5 below shows the determined biomass when 350 μl of a single substrate is supplied and the predicted biomass attained from the supply of a particular volume of that same substrate as found in the BTEXS mixture.

For example, P. putida F1 achieves a biomass of 0.34 g/l when supplied with benzene as a single substrate (350 μl). The same strain has available to it 126 μl of benzene in the BTEXS mixture. Based on a biomass of 0.34 g/l achieved with 350 μl a predicted biomass of 0.12 g/l is achievable from 126 μl of benzene.

TABLE 5

| | | Biomass single | Biomass |
|---|---|---|---|
| | Substrate | Determined | BTEXS |
| Strain | volume | (Table 1) | Predicted |
| F1 (benzene) | 350 μl | 0.34 g/l | |
| | 126 μl | | 0.122 g/l |
| F1 (toluene)* | 350 μl | 0.72 g/l | |
| (⅔ of 87 μl used by F1) | 58 μl | | 0.119 g/l |
| F1 (Ethylbenzene) | 350 μl | 0.67 g/l | |
| | 11 μl | | 0.023 g/l |
| mt-2 (xylene) | 350 μl | 0.53 g/l | |
| | 8 μl | | 0.012 g/l |
| mt-2 (toluene)* | 350 μl | 0.37 g/l | |
| (⅓ of 87 μl used by mt-2) | 29 μl | | 0.09 g/l |
| CA-3 (styrene) | 350 μl | 0.79 g/l | |
| | 118 μl | | 0.26 g/l |
| Total predicted | | | 0.624 g/l |
| Total observed | | | 1.03 g/l |

*F1 and mt-2 compete for toluene. Based on FIG. 2 strain F1 is predicted to utilize two thirds of the toluene and mt-2 one third.

If all individual predicted values (in bold) are added together the value of 0.624 g/l is obtained. However the actual biomass achieved is 1.03 indicating a synergistic effect.

### (ii) PHA Production

The effect on PHA yield is even more pronounced, as shown in Table 6 below:

TABLE 6

| | | Biomass single | Biomass |
|---|---|---|---|
| | | Determined | (BTEXS) |
| Strain | Substrate volume | (Table 1) | Predicted |
| F1 (benzene) | 350 μl | 0.034 g/l | |
| | 126 μl | | 0.012 g/l |
| F1 (toluene) | 350 μl | 0.14 g/l | |
| | 87 μl | | 0.034 g/l |
| F1 (Ethylbenzene) | 350 μl | 0.074 g/l | |
| | 11 μl | | 0.002 g/l |
| mt-2 (xylene) | 350 μl | 0.12 g/l | |
| | 8 μl | | 0.003 g/l |
| mt-2 (toluene) | 350 μl | 0.063 g/l | |
| | 87 μl | | 0.016 g/l |
| CA-3 (styrene) | 350 μl | 0.22 g/l | |
| | 118 μl | | 0.074 g/l |
| Total predicted | | | 0.141 g/l |
| Total observed | | | 0.21 g/l |

If all individual predicted values (in bold) are added the value of 0.141 g/l is obtained, which is 1.5 fold less from the observed value of 0.21 g/l.

This confirms the synergistic effect for both biomass (CDW values) and PHA when growing the defined mixed culture of Pseudomonas strains (P. putida mt-2, F1 and CA-3) on the mixed substrate (BTEXS).

All references throughout this application are hereby incorporated by reference herein in their entireties, as though individually incorporated by reference, to the extent each reference is (at least partially) not inconsistent with the present disclosure (for example, a reference that is partially inconsistent is incorporated by reference except for the partially inconsistent portion of the reference).

All patents and publications mentioned in the specification reflect the level of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, fermentation devices, starting materials, separation and/or extraction methods, and bacterial strains other than those specifically exemplified can be employed in the practice of the invention without resort to undue experimentation. All art-known functional equivalents, of any such methods, and methods, fermentation devices, starting materials, separation and/or extraction methods, and bacterial strains other than those specifically exemplified are intended to be included in this invention. Whenever a range is given in the specification, for example, a temperature range, a time range, or a composition range, all intermediate ranges and subranges, as well as all individual values included in the ranges given are intended to be included in the disclosure.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the invention.

### REFERENCES CITED

Arvin E, Jensen B K, Gundeersen A T. (1989) Substrate Interactions during Aerobic Biodegradation of Benzene. Applied and Environmental Microbiology 55:3221-3225
Chang, M.-K., T. C. Voice, and C. S. Criddle. 1993. Kinetics of Competitive Inhibition and Cometabolism in the Biodegradation of Benzene, Toluene, and p-Xylene by Two Pseudomonas Isolates. Biotechnology & Bioengineering 41:1057-1065
Munoz, R., L. Diaz, S. Bordel, and S. Villaverde. 2007. Inhibitory effects of catechol accumulation on benzene biodegradation in Pseudomonas putida F1 cultures. Chemosphere 68:244-252
Reardon K F, Mosteller D C, Rogers J D B (2000) Biodegradation Kinetics of Benzene, Toluene, and Phenol as Single and Mixed Substrates for Pseudomonas putida F1. Biotechnology & Bioengineering 69:385-400
Ward P G, Goff M, Donner M, Kaminsky W, O'Connor K (2006) A two step chemo-biotechnological conversion of polystyrene to a biodegradable plastic. Environmental Science and Technology 40:2433-2437

What is claimed is:

1. A method for producing polyhydroxyalkanoate (PHA), said method comprising the steps of:
(i) culturing in a culture medium containing a substrate comprising one or more monoaromatic hydrocarbons selected from the group consisting of benzene, toluene, ethylbenzene, ortho-xylene, meta-xylene, para-xylene and styrene, one or more strains of Pseudomonas putida selected from the group consisting of F1, mt-2 and CA-3 having the respective accession numbers DSM 6899, NCIMB10432 and NCIMB41162; and
(ii) recovering the PHA produced from the culture medium; with the following provisos:
(A) when the substrate comprises benzene, Pseudomonas putida strain F1 is present in the culture medium;
(B) when the substrate comprises toluene, Pseudomonas putida strain(s) F1 and/or mt-2 is/are present in the culture medium;
(C)when the substrate comprises ethylbenzene, Pseudomonas putida strain F1 is present in the culture medium;
(D) when the substrate comprises ortho-, meta- or para-xylene, Pseudomonas putida strain mt-2 is present in the culture medium;
(E) when the substrate comprises styrene, Pseudomonas putida strain CA-3 is present in the culture medium; and
(F) the substrate cannot comprise styrene in the absence of at least one of benzene, toluene, ethylbenzene and ortho-, meta- or para-xylene.

2. The method according to claim 1, wherein the substrate comprises benzene and the one or more strains of Pseudomonas putida comprises F1.

3. The method according to claim 1, wherein the substrate comprises toluene and the one or more strains of Pseudomonas putida comprises F1 or mt-2.

4. The method according to claim 1, wherein the substrate comprises toluene and the one or more strains of Pseudomonas putida comprise F1 and mt-2.

5. The method according to claim 1, wherein the substrate comprises ortho-xylene and the one or more strains of Pseudomonas putida comprises mt-2.

6. The method according to claim 1, wherein the substrate comprises meta-xylene and the one or more strains of Pseudomonas putida comprises mt-2.

7. The method according to claim 1, wherein the substrate comprises para-xylene and the one or more strains of Pseudomonas putida comprises mt-2.

8. The method according to claim 1, wherein the substrate comprises benzene, toluene, ethylbenzene, para-xylene and styrene, and the one or more strains of Pseudomonas putida comprise F1, mt-2 and CA-3.

9. The method according to claim 8, wherein the substrate comprises benzene, toluene, ethylbenzene, para-xylene and styrene in a respective ratio by weight of total substrate of about 10-20:5-15:1-2:0.5-1.5:10-20.

10. The method according to claim 8, wherein the substrate comprises benzene, toluene, ethylbenzene, para-xylene and styrene in a respective ratio by weight of total substrate of about 16:11:1.5:1:15.

11. The method according to claim 1, wherein the PHA recovered from the culture medium comprises at least 80% or at least 85% or at least 90% or at least 95% medium chain length (mcl) PHA.

12. The method according to claim 11, wherein at least 80% or at least 85% or at least 90%or at least 95% of the mclPHA comprises repeating units of C8, C10 and C12 monomers.

13. The method according to claim 12, wherein the mclPHA comprises repeating units of C8, C10 and C12 monomers in a respective amount by weight of mclPHA of 10-25% C8, 60% -75% C10 and 10%-25% C12, wherein the amount of C8, C10 and C12 totals 100%.

14. The method according to claim 13 wherein the mclPHA comprises repeating units of C8, C10 and C12 monomers in a respective amount by weight of mclPHA of 16% C8, 66% C10 and 18% C12.

15. The method according to claim 1, wherein the method comprises culturing the one or more strains of Pseudomonas putida in the culture medium for a period of from about 12 hours to about 72 hours at a temperature of from about 25° C. to about 35° C.

16. The method according to claim 1, wherein the culture medium is nitrogen limited, having a maximum nitrogen content of about 0.5 g/liter culture medium.
